(19) United States
(12) Patent Application Publication
(10) Pub. No.: US 2023/0082130 A1
(43) Pub. Date: Mar. 16, 2023

(54) USE OF CANNABINOID COMPOUNDS TO TREAT OR PREVENT FUNGAL GROWTH OF C. ALBICANS

(71) Applicant: Demetrix, Inc., Berkeley, CA (US)

(72) Inventors: Cynthia W. Byrant, Raleigh, NC (US); Alison Watta, Durham, NC (US)

(21) Appl. No.: 17/939,703

(22) Filed: Sep. 7, 2022

Related U.S. Application Data
(60) Provisional application No. 63/241,516, filed on Sep. 7, 2021.

(51) Int. Cl. A61K 31/05; A61K 9/00; A61P 31/10

(52) U.S. Cl. CPC A61K 31/05; A61K 9/0053; A61P 31/10

(57) ABSTRACT

Methods of treating or preventing the growth of Candida albicans (“C. albicans”) using cannabinoid compounds are described herein. The cannabinoid compounds can include one or more of cannabidivarin (“CBDV”), cannabidiorcol (“CBD-C1”), cannabidol-C₂ (“CBD-C₂”), cannabidol-C₄ (“CBD-C₄”), cannabigerivarin (“CBGV”), cannabidol (“CBD”), and cannabigerol butyl (“CBG-C₄”). The methods can be used to treat or prevent oral thrush, athlete's foot, vaginal (yeast) infections, systemic infections, and other infections. Compositions and articles including the cannabinoid compounds are further disclosed.

### CROSS-REFERENCE TO RELATED APPLICATIONS

The present application claims the priority benefit of U.S. Provisional Patent App. Ser. No. 63/241,516, filed Sep. 7, 2021, which is hereby incorporated herein by reference.

### TECHNICAL FIELD

The present disclosure generally relates to the use of cannabinoid compounds to treat fungal growth of Candida albicans.

### BACKGROUND

Candida albicans (“C. albicans”) is a yeast fungi commonly found in the human gut flora, skin, and occasionally the human oral cavity. Although not typically harmful, C. albicans can cause disease and is the most common fungal human pathogen that does so. Diseases caused by C. albicans can range in severity from relatively mild mucosal infections such as Candidiasis (oral thrush), skin infections such as athlete's foot, and more serious infections including vaginal yeast infections, bladder infections, and systematic infections. Additionally, infections with C. albicans can present issues in medical treatment as it can spread in hospitals and adhere to catheters and other medical implants. Innocuous C. albicans can become pathogenic after disruption of the immune system or disruption of the host environment through, for example, antibiotic treatment. With such disruptions, C. albicans can transition from a commensal fungus to a pathogenic fungus. It is presently estimated that 1.7 billion people worldwide suffer from invasive fungal infections.

### DETAILED DESCRIPTION

Pathogenic infections of C. albicans can cause a variety of diseases in humans that are difficult to control with known treatments. Even mild pathogenic infections of C. albicans are a concern to medical professionals due to the potential of C. albicans to progress into systemic infections which can have mortality rates as high as 50%.

Presently, the standard course of treatment to control C. albicans is treatment with antifungal drugs such as miconazole nitrate, fluconazole or echinocandins. Unfortunately, C. albicans strains with antifungal resistance to miconazole nitrate, fluconazole and echinocandins are becoming more common due to the excessive use of antifungal agents in agriculture as well as other improper uses. In contrast to antibiotics, development of new antifungal therapies is considered difficult because fungal cells operate using the same eukaryotic cell mechanisms as humans limiting the number of fungal-specific mechanisms that can be targeted. Treatment of antifungal resistant strains of C. albicans is extremely difficult.

The present application describes the novel use of one or more cannabinoid compounds to inhibit the growth of C. albicans. The cannabinoid compounds are unrelated to known antifungal compounds and can be used to prevent the formation of pathogenic C. albicans or to treat any existing pathogenic infection of C. albicans. The cannabinoid compounds can be included in various products such as personal care products and systemic care products to treat C. albicans infections.

It has specifically been found that cannabidivarin (“CBDV”), cannabidiorcol (“CBD-C1”), cannabidol-C₂ (“CBD-C₂”), cannabidol-C₄ (“CBD-C₄”), cannabigerivarin (“CBGV”), cannabidol (“CBD”), and cannabigerol butyl (“CBG-C₄”) each exhibit a minimum inhibitory concentration (“MIC”) against C. albicans of 200 82 g/mL or less with CBDV, CBD-C₁, CBD-C₂, and CBD-C₄ each having a MIC of less than 25 μg/mL. Such MIC values means that these cannabinoid compounds are highly effective at inhibiting the growth of C. albicans. Thus, these cannabinoid compounds can be included in various products to prevent, reduce, or eliminate, an infection caused by C. albicans.

Other cannabinoid compounds however were not found to be effective at inhibiting the growth of C. albicans when present at such lower concentrations. For example, each of cannabigerol (“CBG”), cannabigerolic acid (“CBGA”), cannabichromene (“CBC”), cannabidivarinic acid (“CBDVA”), cannabicyclol (“CBL”), cannabinol (“CBN”), cannabidiolic acid (“CBDA”), (+)-cannabidiol (“((+)-CBD”), cannabigerovarinic acid (“CBGVA”), cannabigerolic acid butyl (“CBGA-C₄”), cannabichromenic acid (“CBCA”), cannabicyclolic acid (“CBLA”), and cannabinolic acid (“CBNA”) failed to demonstrate a MIC at concentrations of 200 μg/mL or less.

Prior to the present discovery, it was not appreciated that cannabinoid compounds could exhibit antifungal properties, nevertheless which specific cannabinoid compounds could act to inhibit the growth of C. albicans. By selecting the cannabinoid compounds with the greatest effectiveness against C. albicans, the present disclosure provides improved methods of inhibiting the growth of C. albicans, and methods of preventing, or treating, diseases or conditions caused by the fungus.

The present discovery was facilitated by the Applicant's unique methods of producing hereto rare cannabinoid compounds in appreciable quantities including through chemical synthesis reactions and growth in yeast cultures. Prior to the Applicant's research, the lack of viable production of individual cannabinoid compounds obviated the ability to identify cannabinoid compounds which exhibited antifungal properties against C. albicans. Additional details about the production of producing rare cannabinoid compounds are described in PCT Patent Application Nos. WO 2020/069142 A1, WO 2020/069214 A2, WO 2021/05597 A1; and WO 2020/236789 A1, each of which is incorporated herein by reference.

Prior to the Applicant's process of isolating specific and unique cannabinoid compounds from non-horticultural sources, cannabinoid compounds were extracted and isolated only from naturally grown marijuana plants which drastically limited the volume of the rarer cannabinoid compounds available for research or use. Thus, these non-horticulturally derived cannabinoid compounds offer benefits in regard to the treatment of diseases caused by the fungal growth of C. albicans not previously contemplated. As used herein, non-horticulturally derived cannabinoid compounds refers to cannabinoid compounds not grown in plants (e.g., not through horticulture or agriculture).

Additionally, isolated cannabinoid compounds extracted from marijuana plants can also suffer from purity issues as certain unavoidable containments (such as other natural marijuana plant compounds, irremovable amounts of other cannabinoid compounds, etc.) can remain present in isolated cannabinoid compounds extracted from marijuana plants. Such unavoidable containments can impact the quality of the data or even alter the apparent functioning of the cannabinoid compounds. Compositions and methods of treating C. albicans that use horticulturally derived cannabinoid compounds may not exhibit the same effects as compositions and methods using purer cannabinoid compounds such as the cannabinoid compounds contemplated herein. As can be appreciated however, horticulturally derived cannabinoid compounds can be used in certain embodiments of the disclosure if the horticulturally derived cannabinoid compounds are sufficiently pure and/or if any containments are sufficiently well understood.

Generally, personal care products and systemic care products that can reduce, or eliminate, the growth of C. albicans can include an effective concentration of one or more of CBDV, CBD-C₁, CBD-C₂, CBD-C₄, CBGV, CBD, and CBG-C₄. Personal care products refer to products used for personal care such as hygienic devices, grooming devices, and other self-care devices. Systemic care devices refer to products which treat the entire body such as ingestible compositions. In certain embodiments, the one or more cannabinoid compounds can include CBDV, CBD-C₁, CBD-C₂, and/or CBD-C₄. In certain embodiments, each of the foregoing cannabinoid compounds can be included, while in other embodiments, only a subset of such cannabinoid compounds may be included. In certain embodiments, only a single cannabinoid compound may be included.

The concentration of the selected cannabinoid compound(s) can vary depending on factors such as the desired treatment, the severity of the C. albicans infection, the location and type of infection (e.g., topical or systemic), the duration of treatment, and the method of delivering the cannabinoid compound(s) to a subject. For example, in certain embodiments, treatment of athlete's foot may include a relatively greater amount of the one or more cannabinoid compounds due to the physiology of the foot compared to an oral care product to treat oral thrush having a more sensitive physiology. Treatment of systemic infections can vary depending on the amount of the cannabinoid compounds included in a pill, capsule or food; the frequency the cannabinoid compounds are provided; and the biological half-life of the cannabinoid compounds remaining in the body.

In certain embodiments, the concentration of the cannabinoid compounds in a personal care product can be about equal to the MIC value of the cannabinoid compounds. In other embodiments, the concentration of the cannabinoid compounds can be greater than the MIC value, such as in examples where contact is shorter in duration (e.g., oral care products such as a toothpaste or mouthwash). In certain embodiments, the cannabinoid compounds can be included in the personal care product at a concentration of about 200 μg per mL, or g, of the personal care product or less.

In certain embodiments, the compositions, products, articles, and methods described herein can be substantially or entirely free of cannabinoid compounds other than CBDV, CBD-C₁, CBD-C₂, CBD-C₄, CBGV, CBD, and CBG-C₄. For example, the compositions, products, articles, and methods can be substantially or entirely free of tetrahydrocannabinol (“THC”) in addition to CBG, CBGA, CBC, CBDVA, CBL, CBN, CBDA, (+)-CBD, CBGVA, CBGA-C₄, CBCA, CBLA, and CBNA. As used herein, substantially free can mean less than about 5%, less than about 4%, less than about 3%, less than about 2%, less than about 1%, less than about 0.5%, less than about 0.1%, or less than about 0.01%. In certain embodiments, the cannabinoid compounds can be produced using non-horticulturally derived methods such as through chemical synthesis (e.g., organic synthesis reactions) or through modification of yeast and/or bacterial cells to produce the cannabinoid compounds in high purity. However, in certain embodiments, cannabinoid compounds can also be a natural product, e.g., an extract of a cannabis plant if sufficiently pure. In certain embodiments, substantially pure means that the isolated cannabinoid compound, when added, includes about 3% or less of contaminants, about 2% or less of contaminants, about 1% or less of contaminants, about 0.5% or less of contaminants, about 0.1% or less of contaminants, or about 0.01% or less of contaminants.

In certain embodiments, the compositions, products, articles, and methods described herein can be utilized on a predetermined schedule (e.g., nightly, twice daily, etc.) or can be utilized on an as-needed basis. In certain embodiments, the predetermined schedule can be based on the half-life of the cannabinoid compounds as well as the release dynamics of the cannabinoid compounds.

In certain embodiments, it can be useful to use the compositions, products, articles, and methods described herein as part of routine health care to prophylactically treat against infection with C. albicans. In other embodiments, the personal care and systemic care products described herein can be used only by health professionals (e.g., dentists, doctors, surgeons, etc.) or under their supervision.

For embodiments directed to oral health care (e.g., oral thrush), the cannabinoid compounds can be included in any known oral health care product. For example, the cannabinoid compounds can be included in toothpaste, mouthwash, gargles, solutions, drops, emulsions, suspensions, liquids, gels, lozenges, mints, chewable, wipes, powders, or floss. The oral health care products can be intended for use by consumers and patients and/or be intended for use by dental professionals (e.g., dental hygienists, dentists and oral surgeons).

In certain embodiments, the cannabinoid compounds can be incorporated into an oral care composition, product or device, such as a toothpaste, a tooth gel, powder, wipe, a mouthwash or rinse, or a dental floss. In certain embodiments, the cannabinoid compounds can be included in a separate oral care composition, product or device which will be used separately from other compositions, products and devices employed in the prophylactic oral care regimen. For example, the cannabinoid compounds can be incorporated into a mouthwash or rinse, a gum, a lozenge or a chewable tablet.

In certain embodiments, the cannabinoid compounds can be included in dentifrices such as toothpastes, tooth gels, tooth powders and liquid dentifrices. Toothpastes and tooth gels generally include a dental abrasive, a surfactant, a thickening agent, a humectant, a flavoring agent, a sweetening agent, a coloring agent and water. Toothpastes and tooth gels can also include opacifying agents, anti-caries agents, anti-calculus agents, tooth whitening agents, and other optional ingredients. Liquid dentifrices can comprise water, ethanol, a humectant, a surfactant, a thickening agent, an abrasive, an anti-caries agent, a flavoring agent and a sweetening agent.

In certain embodiments, the cannabinoid compounds can be included in gels such as dentifrice gels, non-abrasive gels and subgingival gels. Non-abrasive gels and subgingival gels generally include a thickening agent, a humectant, a flavoring agent, a sweetening agent, a coloring agent, and water. Such gels can also include one or more anti-caries agents and/or anti-calculus agents.

In certain embodiments, the cannabinoid compounds can be included in creams. The creams can include a thickening agent, a humectant and a surfactant, and can include a flavoring agent, a sweetening agent, and a coloring agent.

In certain embodiments, ointments suitable for oral use or topical application can generally include one or more of the following: fats, oils, waxes, paraffins, silicones, plastibase, alcohols, water, humectants, surfactants, thickening agents, talc, bentonites, zinc oxide, aluminum compounds, preservatives, antiviral compounds, and other ingredients.

In certain embodiments, mouthwashes, rinses, gargles and sprays can include water, ethanol, and/or a humectant, and can also include a surfactant, a flavoring agent, a sweetening agent, and a coloring agent, and can include a thickening agent and one or more anti-caries agents and/or anti-calculus agents.

Suitable oral care devices include materials (such as sutures and sponges), flosses, tapes, chips, strips, fibers, a toothpick or rubber tip, syringes, dental implants and dental appliances (such as trays and troughs that fit over and cover the teeth and, optionally, the periodontal tissue) having one or more cannabinoid compounds adhered to, absorbed into, bound to, attached to, entrapped in, enclosed in, coated onto, or otherwise incorporated into, them.

In certain embodiments, the cannabinoid compounds described herein can be included in a compressed chewable tablet. Such compressed chewable tablets can include a water-disintegrable, compressible carbohydrate (such as mannitol, sorbitol, maltitol, dextrose, sucrose, xylitol, lactose and mixtures thereof), a binder (such as cellulose, cellulosic derivatives, polyvinyl pyrrolidone, starch, modified starch and mixtures thereof), and, optionally, a lubricant (such as magnesium stearate, stearic acid, talc, and waxes), sweetening, coloring and flavoring agents, a surfactant, a preservative, and other ingredients. All of the ingredients, including the cannabinoid compounds, can be dry blended and compressed into a tablet.

In certain embodiments, the cannabinoid compounds described herein can be included in a tooth whitening composition which is applied to the teeth by means of a dental tray or trough. Alternatively, a separate composition comprising the cannabinoid compounds can be applied to the teeth in a cleaned or different tray or trough. In additional or alternative embodiments, a wash or rinse comprising the cannabinoid compounds could be used to rinse the mouth before and/or after the application of the tooth whitening composition or toothpaste. In certain embodiments, the cannabinoid compounds can be applied using a flexible strip

As can be appreciated, chewing gum compositions generally include a gum base, a flavoring agent and a sweetening agent. Suitable gum bases include jelutong, rubber, latex, chicle, and vinylite resins, desirably with conventional plasticizers or softeners. Examples of suitable plasticizers include triacetin, acetyl tributyl citrate, diethyl sebacetate, triethyl citrate, dibutyl sebacetate, dibutyl succinate, diethyl phthalate and acetylated monoglycerides. Typically, chewing gum compositions contain from about 50% to about 99% gum base, from about 0.4% to about 2% of a flavoring agent and from about 0.01% to about 20% of a sweetening agent. The cannabinoid compounds can be incorporated into a gum base by, e.g., stirring them into a warm gum base or coating them onto the outer surface of the gum base.

Methods of contacting tissues of the mouth with oral care compositions are well known in the art. Suitable methods include rinsing the tissue with a solution (e.g., a mouthwash, rinse, spray, liquid dentifrice, or other solution), brushing the teeth with a dentifrice (e.g., a toothpaste, tooth gel, or powder), applying a non-abrasive solution, gel, paste, cream or ointment directly to the tissue (with or without the use of an applicator) (including fluoride varnishes), chewing gum, chewing or sucking a lozenge, mint or tablet, and many other means of topical application. Suitable applicators for applying oral care compositions, such as solutions, gels, pastes, creams and ointments, to a tissue include a swab, a stick, a plastic paddle, a dropper, a syringe, a strip, a finger, or a dental tray or appliance which allows for immersion of the teeth and, optionally, the periodontal tissue in, e.g., a gel or solution thereof In certain embodiments, oral care compositions further comprise, for instance, sutures can be used to close a surgical wound or a wound resulting from a tooth extraction, dental floss can be used to floss the teeth, etc. The treatment of the tissue can be prophylactic treatment. For instance, the tissue may be treated as part of a prophylactic oral care regimen.

Tissues may also be treated prophylactically in connection with a variety of dental procedures, including surgeries and tooth extractions. For instance, the tissue(s) on which surgery is being performed, those tissues near the area where the surgery is being performed or, for ease of treatment, all or substantially of the tissues of the mouth, can be treated prior to surgery, during surgery, after the surgery, or combinations thereof. Similarly for a tooth extraction, the tissue(s) surrounding the tooth which is to be extracted, adjacent tissues or, for ease of treatment, all or substantially of the tissues of the mouth, can be treated prior to tooth extraction, during the tooth extraction, after the tooth extraction, or combinations thereof. For instance, the mouth could be rinsed prior to surgery or tooth extraction with a solution comprising one or more cannabinoid as discussed herein, the wound(s) caused by the surgery or tooth extraction could be closed with sutures having one or more cannabinoid as discussed herein incorporated into them, and/or the mouth could be rinsed immediately after the surgery or tooth extraction, and/or at intervals thereafter, with a solution comprising one or more cannabinoid as discussed herein.

In certain embodiments, a personal care product can alternatively be a topical product to treat C. albicans infections such as athlete's foot, skin infections, or vaginal (yeast) infections. In such embodiments, the one or more cannabinoid compounds can be included in a lotion, a gel, a liquid, a powder, or other personal care composition as known in the art. As can be appreciated, such products can be similar to certain oral care products previously described such as a lotion or a cream previously described. Certain such products can optionally include one or more of an emollient, a moisturizer, and a topical anesthetic to treat any accompanying pain or dryness caused by the C. albicans infection.

In certain embodiments, the cannabinoid compounds described herein can be applied topically by immersion in a composition containing the cannabinoid compounds. For example, the water in a bathtub containing the dissolved cannabinoid compounds described herein can therapeutically treat skin infections caused by C. albicans. In certain embodiments, the water can be treated by dissolving a solid or liquid product, such as a bath salt or bath soap, containing the cannabinoid compounds. In certain embodiments, such products can additionally contain adjunct ingredients such as clays, oatmeal, baking soda, and other known adjunct ingredients.

In certain embodiments, the one or more cannabinoid compounds described herein can be incorporated into a device for treatment, or prevention, of a C. albicans infection. For example, medical devices, wound dressings, catheters, socks, undergarments, casts, slings, pessaries, suppositories, pre-moistened wipes, etc. can each be impregnated, or cleaned, with the one or more cannabinoid compounds described herein. Such devices can be used to either treat a C. albicans infection or to prevent infection. For example, cleaning of medical devices with the cannabinoid compounds described herein can be useful to limit the spread of C. albicans onto, or through, such medical devices.

In certain embodiments, the one or more cannabinoid compounds can alternatively, or additionally, be provided systemically. In such embodiments, the cannabinoid compounds can be included in pills or capsules that can be taken quickly and efficiently on a regular or as needed basis (daily, with meals, etc.).

As can be appreciated, pills and capsules can contain a number on inactive ingredients as known in the art such as dicalcium phosphate dehydrate, microcrystalline cellulose, stearic acid, silicon dioxide, croscarmellose sodium, magnesium stearate, and pharmaceutical glaze. Other known pills and capsules are also contemplated herein. As an additional example, a compressed chewable tablet can include a water-disintegrable, compressible carbohydrate (such as mannitol, sorbitol, maltitol, dextrose, sucrose, xylitol, lactose and mixtures thereof), a binder (such as cellulose, cellulosic derivatives, polyvinyl pyrrolidone, starch, modified starch and mixtures thereof), the cannabinoid compounds and, optionally, a lubricant (such as magnesium stearate, stearic acid, talc, and waxes), sweetening, coloring and flavoring agents, a surfactant, a preservative, and other ingredients. All of the ingredients, including the one or more cannabinoid compounds, are dry blended and compressed into a tablet.

In certain embodiments, the cannabinoid compounds can alternatively be systematically administered to individuals via food products and other comestibles. By way of illustration and not as a limitation, the selected cannabinoid compounds can be incorporated into a beverage, a “smoothie” (fruit, vegetable, nut oil, or yogurt based), a frozen desert (e.g., ice cream or sorbet), a food bar, a nutrition bar, a dressing, a snack, into a flour- or flour-alternative-based product, a rice-based product, pastes, gels, powders, gums, etc. Incorporation into food products can facilitate consumption of the cannabinoid compounds and increase palatability.

As can be appreciated, the exact nature of the food article can influence the bioavailability of the cannabinoid compounds. For example, a cannabinoid included in a large food article may take more time to become bioavailable than the same amount of cannabinoid compounds in a single pill or capsule. Generally, the remainder of the composition or article can constitute any suitable non-bioactive component such as filler, food, or water.

In certain embodiments, the compositions or articles including the cannabinoid compounds described herein can include indicia and/or packaging to convey to end users the amount of the cannabinoid compounds contained therein. For example, a small nutrient bar may be individually labeled and packaged to express to the end user that only a single bar should be consumed.

As will be appreciated, a wide variety of different compositions and articles can be prepared which include the one or more cannabinoid compounds of the present disclosure including compositions and articles not listed here. All such compositions and articles are contemplated herein as they are within the ordinary skill of artisans based on the guidance provided in the present disclosure.

Generally, all of the compositions and articles described herein can be manufactured and produced as known in the art. For example, in certain embodiments, the cannabinoid compounds can be dissolved in a suitable solvent such as an alcohol or oil and then added to the composition or article.

### EXAMPLES

To determine the ability of the cannabinoid compounds described herein to reduce, inhibit, or eliminate, C. albicans a minimum inhibitory concentration (“MIC”) assay was performed. The MIC assay determines at what concentration the cannabinoid compounds inhibit the growth of C. albicans. The assay was performed by Charles River Laboratories International (Wilmington, Mass.) in accordance with industry standards. The results are depicted in Table 1.

TABLE 1

| Cannabinoid | MIC (μg/mL) |
|---|---|
| CBG | >200 |
| CBGA | >200 |
| CBC | >200 |
| CBDVA | >200 |
| CBL | >200 |
| CBN | >200 |
| CBDV | 12.5 |
| CBDA | >200 |
| CBD | 200 |
| CBD-C1 | 25 |
| CBD-C2 | 12.5 |
| CBD-C4 | 12.5 |
| (+)-CBD | >200 |
| CBGV | 50 |
| CBGVA | >200 |
| CBG-C4 | 200 |
| CBGA-C4 | >200 |
| CBCA | >200 |
| CBLA | >200 |
| CBNA | >200 |

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in the document shall govern.

The foregoing description of embodiments and examples has been presented for purposes of description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent articles by those of ordinary skill in the art. Rather it is hereby intended the scope be defined by the claims appended hereto.

It should be understood that certain aspects, features, structures, or characteristics of the various embodiments can be interchanged in whole or in part. Reference to certain embodiments mean that a particular aspect, feature, structure, or characteristic described in connection with certain embodiments can be included in at least one embodiment and may be interchanged with certain other embodiments. The appearances of the phrase “in certain embodiments” in various places in specification are not necessarily all referring to the same embodiment, nor are certain embodiments necessarily mutually exclusive of other certain embodiments. It should also be understood that the steps of the methods set forth herein are not necessarily required to be performed in the orders described, and the order of the steps of such methods should be understood to be merely exemplary. Likewise, additional steps can be included in such methods, and certain steps may be omitted or combined, in methods consistent with certain embodiments.

What is claimed is:

1. A personal care composition comprising one or more cannabinoid compounds, the personal care composition inhibiting the growth of Candida albicans (“C. albicans”); the one or more cannabinoid compounds having a minimum inhibitory concentration (“MIC”) against C. albicans of about 200 μg/mL or less.

2. The personal care composition of claim 1, wherein the one or more cannabinoid compounds comprise cannabidivarin (“CBDV”), cannabidiorcol (“CBD-C1”), cannabidol-C₂ (“CBD-C₂”), cannabidol-C₄ (“CBD-C₄”), cannabigerivarin (“CBGV”), cannabidol (“CBD”), and cannabigerol butyl (“CBG-C₄”).

3. The personal care composition of claim 1, wherein the one or more cannabinoid compounds have a MIC against C. albicans of about 100 μg/mL or less.

4. The personal care composition of claim 1, wherein the one or more cannabinoid compounds have a MIC against C. albicans of about 50 μg/mL or less.

5. The personal care composition of claim 1, wherein the one or more cannabinoid compounds have a MIC against C. albicans of about 25 μg/mL or less.

6. The personal care composition of claim 1, wherein the one or more cannabinoid compounds have a MIC against C. albicans of about 10 μg/mL or less.

7. The personal care composition of claim 1, wherein the one or more cannabinoid compounds have a MIC against C. albicans of about 6.25 μg/mL or less.

8. The personal care composition of claim 1, wherein the one or more cannabinoid compounds have a MIC against C. albicans of about 3.125 μg/mL or less.

9. The personal care composition of claim 1, wherein the one or more cannabinoid compounds have a MIC against C. albicans of about 1 μg/mL or less.

10. The personal care composition of claim 1 is a liquid, gel, powder, or cream.

11. The personal care composition of claim 1 is effective in treating oral thrush, athlete's foot, or a yeast infection.

12. An ingestible article comprising one or more cannabinoid compounds, the ingestible article inhibiting the growth of Candida albicans (“C. albicans”); the one or more cannabinoid compounds having a minimum inhibitory concentration (“MIC”) against C. albicans of about 200 μg/mL or less.

13. The ingestible article of claim 12, wherein the one or more cannabinoid compounds comprise cannabidivarin (“CBDV”), cannabidiorcol (“CBD-C1”), cannabidol-C₂ (“CBD-C₂”), cannabidol-C₄ (“CBD-C₄”), cannabigerivarin (“CBGV”), cannabidol (“CBD”), and cannabigerol butyl (“CBG-C₄”).

14. The ingestible article of claim 12 is a pill, a capsule, or a food article.

15. The ingestible article of claim 12 is a systemic care product.

16. A method of treating or preventing a Candida albicans (“C. albicans) infection, the method comprising administering a one or more cannabinoid compounds to a subject.

17. The method according to claim 16, wherein the one or more cannabinoid compounds comprise cannabidivarin (“CBDV”), cannabidiorcol (“CBD-C1”), cannabidol-C₂ (“CBD-C₂”), cannabidol-C₄ (“CBD-C₄”), cannabigerivarin (“CBGV”), cannabidol (“CBD”), and cannabigerol butyl (“CBG-C₄”).

18. The method of claim 16, wherein the one or more cannabinoid compounds are administered to the subject through a medical device.

19. The method of claim 16, wherein the one or more cannabinoid compounds are administered to infected tissue of the subject.

20. The method of claim 16, wherein the one or more cannabinoid compounds reduce the amount of Candida albicans (“C. albicans”) present in the infected tissue.
